(19) United States
(12) Patent Application Publication
(10) Pub. No.: US 2007/0299518 A1
(43) Pub. Date: Dec. 27, 2007

(54) Device with nanocomposite coating for controlled drug release

(75) Inventor: Patrick H. Ruane, Redwood City, CA (US)

Correspondence Address: BRINKS HOFER GILSON & LIONE/CHICAGO/COOK, PO BOX 10395, CHICAGO, IL, 60610, US

(73) Assignee: Med Institute, Inc., West Lafayette, IN (US)

(21) Appl. No.: 11/698,407

(22) Filed: Jan. 26, 2007

Related U.S. Application Data
(60) Provisional application No. 60/762,922, filed on Jan. 27, 2006.

(51) Int. Cl. A61F 2/02

(52) U.S. Cl. 623/11.11

(57) ABSTRACT

An implantable medical device including a nanocomposite coating deposited on at least a portion of a surface of at least one structural element of the device to provide a controlled release of a bioactive agent in one or more dosages is described. The nanocomposite coating includes a matrix, a bioactive agent and inorganic particles. The inorganic particles respond to a stimulus, preferably by generating heat. The response of the particles to the stimulus causes the matrix of the nanocomposite coating to undergo a volume change by, for example, contracting or swelling, thereby releasing at least a portion of the bioactive agent. A method of providing a controlled release of a bioactive agent from a nanocomposite coating on an implantable medical device is described. A method for providing a nanocomposite coating for the controlled release of a bioactive agent on the implantable medical device is also described.

### RELATED APPLICATIONS

The present patent document claims the benefit of the filing date under 35 U.S.C. §119(e) of Provisional U.S. Patent Application Ser. No. 60/762,922, filed Jan. 27, 2006, which is hereby incorporated by reference.

### TECHNICAL FIELD

The present invention relates generally to medical devices and more particularly to coated implantable medical devices for the controlled release of bioactive agents.

### BACKGROUND

Conventional tablet formulations of pharmaceuticals have a less than ideal drug delivery profile. Typically, the pharmaceutical is rapidly and uncontrollably released from the tablet formulation, ultimately reaching a concentration level in the bloodstream that may exceed a toxic threshold value. The concentration level then exponentially decreases over time to an ineffective level, at which point another dosage must be administered. To more effectively and safely deliver pharmaceuticals to treat various ailments, controlled release drug formulations have been developed. These are usually intended to provide a delayed or constant release of the pharmaceutical over an extended time period. A well-known example is extended-release drug capsules used to treat cold or allergy symptoms. In these formulations the pharmaceutical may be enclosed within a polymeric capsule through which it passes by diffusion, as discussed, for example, in U.S. Pat. No. 3,279,996.

A wide range of controlled release technologies has been explored over the past couple of decades, including, more recently, delivery systems based on nanoscale particles. U.S. Pat. No. 6,645,517 B2, for example, discloses a subcutaneously implanted composite of metal “nanoshells” dispersed in a temperature-sensitive polymer, which is also loaded with a pharmaceutical. When the nanoshells are exposed to near-infrared light generated by a laser outside the body, the light exposure causes the nanoshells to generate heat, which in turn causes the polymer to contract and release the pharmaceutical.

As the technology for the controlled release of pharmaceuticals has advanced, interest has shifted to the development of implantable medical devices having controlled release capabilities that can be conveyed to targeted locations in the body for site-specific drug delivery.

For example, it may be possible to treat or mitigate restenosis or thrombosis formation within a blood vessel by controllably releasing a pharmaceutical from an implantable stent or valve. U.S. Pat. No. 6,774,278, for example, discloses a coated implantable medical device having a polymeric porous layer through which a bioactive agent may be controllably released. Other devices coated with a drug-eluting layer have emerged as well. Such devices typically provide a substantially continuous release of the bioactive agent at a specific site in the body.

For the treatment of some conditions, it would be desirable to be able to control the release of the pharmaceutical in a noncontinuous fashion, providing in effect multiple dosages of a pharmaceutical from an implantable device. U.S. Pat. No. 6,524,274, for example, discloses a thermal catheter including an expandable balloon portion coated with a temperature-sensitive polymer that contains a bioactive agent. The thermal catheter is also equipped with electrodes for heating the polymer. When the polymer is heated, it contracts and releases the bioactive agent; upon cooling, the polymer returns to its initial volume and the release of the bioactive agent is halted. It would be desirable to be able to trigger the release of the bioactive agent from the polymer using a heat source which is internal to the polymer. This would allow the heat to be localized to the polymer, thereby avoiding potential damage to adjacent tissue and increasing the efficiency of the process. It further would be desirable to be able to control such an internal heat source from outside the body.

By developing technology for the controlled release of a bioactive agent in multiple dosages from an implantable medical device, it may be possible to optimize the benefit of the bioactive agent to the patient over the desired treatment period. Existing methods and devices do not provide a satisfactory means of controllably and safely initiating and halting the release of the bioactive agent from an implantable medical device in order to provide a controlled release of a pharmaceutical to a specific site in the body.

### BRIEF SUMMARY

The present invention describes an implantable medical device having a nanocomposite coating for the controlled release of a bioactive agent. The medical device performs a function when implanted within an animal and may overcome the limitations of existing devices for providing a controlled release of a bioactive agent in one or more dosages at a particular site in the body. The present invention also describes a method of providing a controlled release of a bioactive agent from the nanocomposite coating on the implantable medical device, and a method of disposing the nanocomposite coating on the implantable medical device for the controlled release of a bioactive agent.

According to one embodiment, the implantable medical device has a nanocomposite coating deposited on at least a portion of a surface of at least one structural element of the device. The nanocomposite coating includes a matrix, a bioactive agent, and inorganic particles that are responsive to a stimulus. When the inorganic particles are exposed to the stimulus, at least a portion of the bioactive agent is released from the nanocomposite coating.

According to another embodiment, the implantable medical device has a nanocomposite coating deposited on at least a portion of a surface of at least one structural element of the device. The nanocomposite coating includes a hydrogel, a bioactive agent, and metal nanoshells that are responsive to electromagnetic radiation. When the metal nanoshells are exposed to electromagnetic radiation, at least a portion of the bioactive agent is released from the nanocomposite coating.

According to one embodiment, a method of obtaining a controlled release of a drug from a medical device having a function when implanted within an animal includes the steps of: inserting into a body lumen an implantable medical device comprising a nanocomposite coating, which includes a matrix, a bioactive agent, and inorganic particles that are responsive to a stimulus, on at least a portion of a surface of at least one structural element of the device; and then exposing the inorganic particles to the stimulus so that at least a portion of the bioactive agent is released from the nanocomposite coating.

According to one embodiment, a method for providing a coating for the controlled release of a bioactive agent on a medical device having a function when implanted within an animal includes the steps of: preparing a coating formulation comprising a matrix precursor and inorganic particles that are responsive to a stimulus; and depositing the coating formulation onto at least a portion of a surface of at least one structural element of an implantable medical device to form a coated implantable medical device having a nanocomposite coating.

According to another embodiment, a method for providing a coating for the controlled release of a bioactive agent on a medical device having a function when implanted within an animal includes the steps of: preparing a first coating formulation comprising a first matrix precursor and inorganic particles, the inorganic particles being responsive to a stimulus; preparing a second coating formulation comprising a second matrix precursor and a bioactive agent; sequentially depositing the first coating formulation and the second coating formulation onto at least a portion of a surface of at least one structural element of an implantable medical device, thereby forming a coated implantable medical device having a nanocomposite coating.

### BRIEF DESCRIPTION OF THE DRAWINGS

FIG. 1 is a cross-sectional schematic of a coated medical device according to one embodiment (A) before and (B) during exposure to a stimulus.

FIG. 2 is a cross-sectional schematic of a coated medical device according to another embodiment (A) before and (B) during exposure to a stimulus.

### DEFINITIONS

The term “nanocomposite coating” as used herein refers to a coating having an essentially continuous matrix and discrete particles dispersed within at least a portion of the matrix. Preferably, the particles are less than about 1,000 nanometers in size.

The term “bioactive agent” as used herein refers to any pharmaceutically active agent that results in an intended therapeutic effect on the body to treat or prevent conditions or diseases. The terms “therapeutic agent,” “pharmaceutical” and “drug” may be taken to have the same meaning as “bioactive agent” and thus the terms may be used interchangeably.

The term “stimulus” as used herein refers to something that elicits a response from the inorganic particles of the invention.

The term “animal” as used herein refers to a multicellular organism of the kingdom Animalia, including humans and animals.

By “pharmaceutically acceptable salt” is meant those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharm Sciences, 66: 1-19 (1977), which is hereby incorporated by reference.

The term “pharmaceutically acceptable ester” as used herein refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters includes formates, acetates, propionates, butyates, acrylates and ethylsuccinates.

The term “pharmaceutically acceptable prodrug” as used herein refers to those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to provide the parent compound having the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

### DETAILED DESCRIPTION

An implantable medical device with a nanocomposite coating deposited on a portion of a surface thereof to provide a controlled release of a bioactive agent in one or more dosages at a particular site in the body is described. The medical device has a structure including at least one structural element, as will be further described below.

The nanocomposite coating of the invention includes a matrix, a bioactive agent and inorganic particles. The inorganic particles respond to a stimulus, preferably by generating heat. The response of the particles to the stimulus causes the matrix of the nanocomposite coating to undergo a volume change by, for example, contracting or swelling, thereby releasing at least a portion of the bioactive agent.

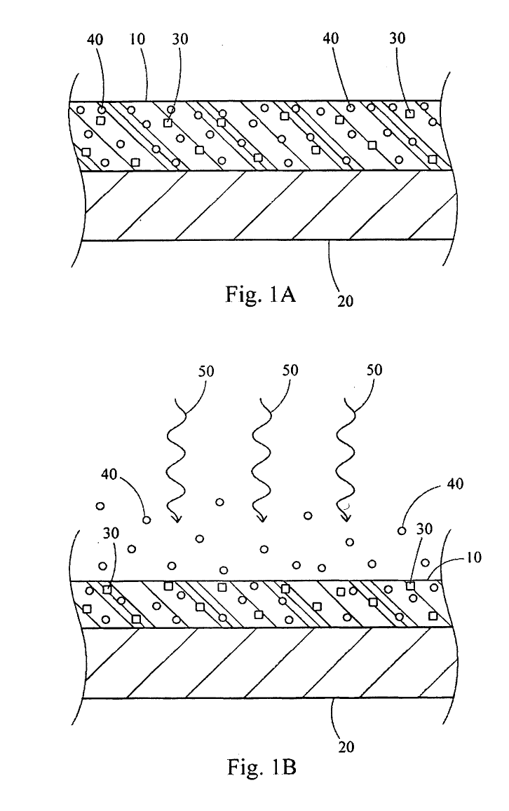

FIG. 1A shows a cross-sectional schematic of the nanocomposite coating 10 deposited on at least a portion of a surface of a structural element 20 of a medical device according to one embodiment. The inorganic particles 30 and bioactive agent 40 are dispersed in the nanocomposite coating 10. When the stimulus 50 is applied, as shown in FIG. 1B, the matrix of the nanocomposite coating 10 contracts and releases the bioactive agent 40.

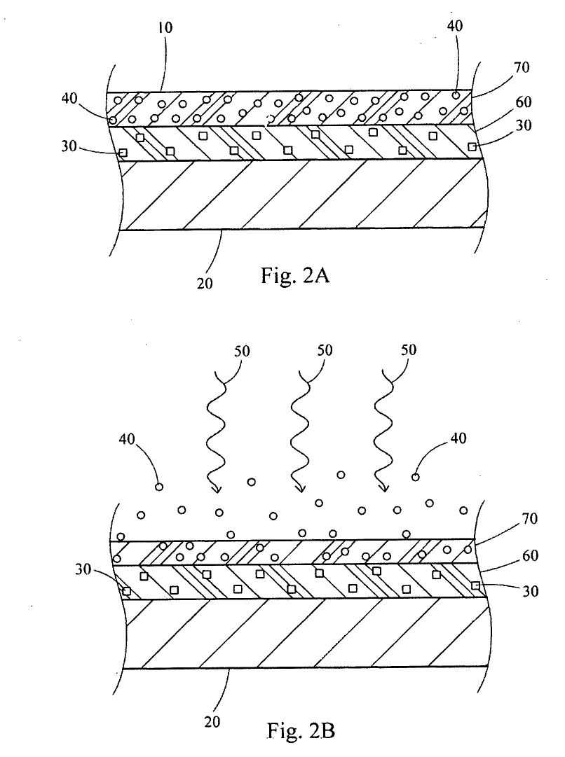

FIG. 2A shows a cross-sectional schematic of the nanocomposite coating 10 deposited on at least a portion of a surface of a structural element 20 of a medical device according to another embodiment. In this embodiment, the nanocomposite coating includes two layers 60 70. The inorganic particles 30 are dispersed in one layer 60, and the bioactive agent 40 is dispersed in the other layer 70. FIG. 2B shows the volume change that occurs when the nanocomposite coating 10 is exposed to the stimulus 50, facilitating release of the bioactive agent 40.

In some embodiments, the change in volume of the matrix may be reversible. That is, when the stimulus is removed, the matrix may return to its initial volume, thereby halting release of the bioactive agent. Such reversible behavior may allow for multiple dosages of a bioactive agent to be released over time, with each dosage commencing when the inorganic particles are exposed to the stimulus and concluding when the stimulus is removed. In other embodiments, the volume change may be controlled, but not reversible. That is, the matrix of the nanocomposite coating may undergo a volume change when the inorganic particles are exposed to the stimulus. but the matrix may not return to its initial volume when the stimulus is removed. Subsequent applications of the stimulus may result in further changes in volume and promote further release of the bioactive agent.

The matrix of the nanocomposite coating preferably includes a polymer. Any polymer that undergoes a change in volume in response to heat may be suitable for use as the matrix of the nanocomposite coating. Preferably, the contraction or swelling of the polymer in response to heat may be reversible. In some embodiments, the polymer may be biodegradable; that is, the polymer may degrade over time under physiological conditions.

Preferably, the polymer may be a hydrogel. Examples of hydrogels that may be used include, without limitation, polyethylene oxide and its copolymers, polyvinylpyrrolidone and its derivatives, hydroxyethylacrylates or hydroxyethyl(meth)acrylates, polyacrylic acids, polyacrylamides, polyethylene maleic anhydride and its derivatives.

According to one embodiment, the hydrogel may contract in response to heat, thereby releasing at least a portion of the bioactive agent. Preferably, the hydrogel may include poly(N-isopropylacrylamide). Poly(N-isopropylacrylamide) reversibly contracts when its temperature is raised above its lower critical solution temperature, or LCST. The LCST of poly(N-isopropylacrylamide) may be only a few degrees above body temperature. When the hydrogel contracts, it may expel at least a portion of the bioactive agent out of the nanocomposite coating.

According to another embodiment, the hydrogel may swell in response to heat, thereby releasing at least a portion of the bioactive agent from the nanocomposite coating. A hydrogel according to this embodiment may be, for example, a poly(acrylamide)-poly(acrylic acid) or a photopolymerized (photocrosslinked) acrylated polypropylene oxide-polyethylene oxide block copolymer.

In some embodiments, the matrix of the nanocomposite coating may include two or more layers. Each layer preferably includes a polymer. Each layer may further include a bioactive agent and/or inorganic particles. In one embodiment, the two or more layers may include the same polymer. In another embodiment, the two or more layers may include different polymers. In yet another embodiment, the two or more layers may include a combination of same and different polymers. The layers preferably may include hydrogels as described above, although other polymers also may be used.

The nanocomposite coating may have a thickness of from about 0.1 micron to about 100 microns. Preferably, the nanocomposite coating may have a thickness of from about 1 micron to 50 microns. More preferably, the nanocomposite coating may have a thickness of from about 5 microns to about 25 microns. In some embodiments, the nanocomposite coating may have a biocompatible layer disposed thereon.

The inorganic particles are dispersed in at least a portion of the matrix. Preferably, the concentration of inorganic particles in the matrix is sufficient to cause the matrix to undergo a volume change. The concentration range can vary widely, but typically is less than about 30% by volume. More typically, the concentration of particles in the matrix is less than about 20%, 10%, 5%, or 1% by volume. It is also envisioned that the concentration of particles in the matrix may be less than about 0.1% by volume, or less than about 0.01% by volume.

In some embodiments, the stimulus may be an external stimulus, that is, a stimulus generated by a source present outside the body. In other embodiments, the stimulus may be an internal stimulus, that is, a stimulus generated by a source introduced into the body, such as, for example, an endovascular laser. Preferably, the inorganic particles respond to the stimulus by generating heat.

According to one embodiment, the stimulus may be a magnetic field. According to this embodiment, at least a portion of each inorganic particle may be formed of a magnetically-responsive material. As defined herein, particles formed of a magnetically-responsive material respond to a magnetic field, preferably by generating heat. The magnetically-responsive material may contain at least one element selected from the group consisting of Fe, Co, Cr, Mo, Mn, and Ni. Preferably, the magnetically-responsive material may be iron oxide. Preferred iron oxides include magnetite (Fe₃O₄) and maghemite (γ-Fe₂O₃) because of their biocompatibility and their generally appropriate magnetic properties. The amount of heat generated in response to the magnetic field may depend on the size, structure, composition, and concentration of the inorganic particles in the nanocomposite coating.

Preferably, the magnetic field may be an alternating current (ac) magnetic field. The magnetic field may be generated by, for example, a magnetic resonance imaging (MRI) device. Generally, to avoid deleterious physiological responses, ac magnetic field frequencies (f) within the range of from about 0.05 MHz to about 1.2 MHz and magnetic field amplitudes (H) of up to about 15 kA/m may be employed (Q. A. Pankhurst et al., J. Phys. D. Appl. Phys. 36 (2003) R167-R181). It has been reported that exposure to magnetic fields having a product H•f that does not exceed 4.85×10⁸ A m⁻¹ s⁻¹ is safe and tolerable (Atkinson et al., IEEE Trans. Biomed. Eng. 9, 549-56 (1983)).

In another embodiment, the stimulus may be electromagnetic radiation (light). According to this embodiment, at least a portion of each inorganic particle may be formed of a photo-responsive material. As defined herein, a photo-responsive material responds to electromagnetic radiation (photons), preferably by generating heat. The photo-responsive material may contain at least one element selected from the group consisting of Au, Ag, Pt, Pd, Ir, Rh, Ru, Os, Re, Tc, W, Ta, Nb, Hf, Zr, Y, Sc, Ti, V, Cr, Mo, Mn, Tc, Fe, Co, Ni, Cu, Zn, Cd, Al, Ga, In, Tl, Si, Ge, Sn, Pb, Bi, Sb, As, Se, Te, Po, Ce, Pr, Nd, Sm, Eu, Gd, Tb, Dy, Ho, Er, Tm, Yb, and Lu. Preferably, the photo-responsive material may be gold or silver. Inorganic particles formed at least in part of gold or silver may be biocompatible and have generally appropriate optical properties. The inorganic particles may be designed to strongly absorb electromagnetic radiation at a predetermined wavelength for conversion into heat. The amount of heat generated in response to the electromagnetic radiation may depend on the size, structure, composition, and concentration of the inorganic particles in the nanocomposite coating.

Preferably, the electromagnetic radiation may have a wavelength in the near-infrared (IR) portion of the spectrum. In particular, the electromagnetic radiation may have a wavelength in the range of from about 700 to 2500 nm. Preferably, the electromagnetic radiation may have a wavelength in the range of from about 800 nm to 1200 nm. Light in this wavelength range may pass through human tissue with a small amount of attenuation. Preferably, the light may be generated by a laser, such as, for example, a Nd:YAG laser emitting at a wavelength of 1064 nm. Alternatively, the light may be generated by a light-emitting diode (LED). Near-IR light in controlled doses is generally considered to be safe for repeated exposures.

Preferably, the inorganic particles may generate enough heat in response to the stimulus to cause the nanocomposite coating to contract or swell, thereby causing at least a portion of the bioactive agent to be released from the nanocomposite coating. It is further preferable that the heat generated by the particles is insufficient to cause damage to body tissue. Preferably, the inorganic particles may generate enough heat to raise the temperature of at least a portion of the nanocomposite coating to between about 1 degree and about 30 degrees above body temperature (i.e., 37° C.); that is, the temperature of at least a portion of the nanocomposite coating may lie within the range of about 38° C. and about 67° C. Even more preferably, the temperature of at least a portion of the nanocomposite coating may be raised between about 1 degree and about 20 degrees above body temperature; that is, the temperature of at least a portion of the nanocomposite coating may lie within the range of about 38° C. and about 57° C. Most preferably, the temperature may be raised between about 1 degree and about 15 degrees above body temperature; that is, the temperature of at least a portion of the nanocomposite coating may lie within the range of about 38° C. and about 52° C.

When the particles are exposed to the stimulus, the rate of release, or release kinetics, of the bioactive agent(s) from the nanocomposite coating may be determined by a variety of factors, including characteristics of the bioactive agent, the type of binding of the bioactive agent within the nanocomposite coating, the chemistry and structure of the nanocomposite coating, the duration of the exposure, and the temperature in the vicinity of the bioactive agent.

The size of the particles may be about 1,000 nm or less. Preferably, the size of the particles may be from about 1 nm to 100 nm. Particles of about 100 nm or less in size are commonly referred to as nanoscale particles, nanoparticles, or nanocrystals. More preferably, the size of the particles may be from about 1 to 50 nm. Particles within this size range may provide the desired properties (e.g., optical or magnetic properties) and serve as effective heat emitters due to their high surface area to volume ratio.

The shape of the inorganic particles may be, for example, substantially spherical, semispherical, cylindrical, acicular, cubic, pyramidal, conical, disk-like or plate-like.

The inorganic particles may have a core-shell structure, including a core and an outer layer surrounding the core. Such particles may be referred to as nanoshells. A core-shell structure may impart certain advantages to the particles. For example, a core-shell structure may improve the response of the inorganic particles to the stimulus. A core-shell structure may also improve the biocompatibility of the particles, serve a protective function, facilitate the binding of functional groups onto the particle surface, and/or provide other advantages. In general, the outer layer of particles having a core-shell structure may be formed of at least one or more elements selected from the group consisting of C, Au, Ag, Pt, Pd, Ir, Rh, Ru, Os, Re, Tc, W, Ta, Nb, Hf, Zr, Y, Sc, Ti, V, Cr, Mo, Mn, Tc, Fe, Co, Ni, Cu, Zn, Cd, Al, Ga, In, Tl, Si, Ge, Sn, Pb, Bi, Sb, As, Se, Te, Po, Ce, Pr, Nd, Sm, Eu, Gd, Tb, Dy, Ho, Er, Tm, Yb, and Lu, and the core may be formed of at least one or more elements selected from the group consisting of C, Au, Ag, Pt, Pd, Ir, Rh, Ru, Os, Re, Tc, W, Ta, Nb, Hf, Zr, Y, Sc, Ti, V, Cr, Mo, Mn, Tc, Fe, Co, Ni, Cu, Zn, Cd, Al, Ga, In, Tl, Si, Ge, Sn, Pb, Bi, Sb, As, Se, Te, Po, Ce, Pr, Nd, Sm, Eu, Gd, Tb, Dy, Ho, Er, Tm, Yb, and Lu.

In one embodiment, the outer layer of the particles may be formed of a conductive material and the core may be formed of a dielectric or nonconductive material. Such particles may be referred to as metal nanoshells, due to the use of a conductive outer layer, or shell. This structure may improve the response of the inorganic particles to a stimulus, in particular, to electromagnetic radiation. By adjusting the thickness of the outer layer and the size of the core, the wavelength of electromagnetic radiation absorbed by the particles may be tailored to a specific range or value, as further discussed in U.S. Pat. No. 6,645,517 B2, which is incorporated herein by reference. For example, nanoshells comprising a core of from about 5 nanometers (nm) to about 100 nm in size and an outer layer of from about 1 nm to about 20 nm in thickness may be used. Even more preferably, the core may have a size in the range of from about 5 nm to about 50 nm, and the outer layer may have a thickness in the range of from about 1 nm to about 10 nm. The core layer is preferably formed from gold sulfide or silicon dioxide, and the outer layer is preferably formed of gold. Such particles may be referred to as gold nanoshells.

In another embodiment, the core of the inorganic particles may be formed of an inorganic material, such as, for example, iron oxide, and the outer layer may be formed of an organic material, such as, for example, dextran. Other combinations of an inorganic core material and an organic outer layer material may be used also.

The surface of each inorganic particle further may be chemically functionalized to bind or tether the inorganic particles to the matrix. Such binding may facilitate effective heat transfer between the inorganic particles and the matrix. It also may inhibit loss of the inorganic particles as the matrix contracts or swells in response to the generated heat. The chemical functionalization strategy may depend on the specific type of particle and matrix used to form the nanocomposite coating. A molecule with a thiol group and an acrylate group (e.g., Cys-PEG-acrylate) may be used, for example, as discussed in U.S. Pat. No. 6,645,517.

A bioactive agent may be dispersed in at least a portion of the matrix of the nanocomposite coating. Bioactive agents that may be used in the present invention include, but are not limited to, pharmaceutically acceptable compositions containing any of the bioactive agents or classes of bioactive agents listed herein, as well as any salts, prodrugs, esters and/or pharmaceutically acceptable formulations thereof. Table 1 below provides a non-exclusive list of classes of bioactive agents and some corresponding exemplary active ingredients.

TABLE 1

| Class | Exemplary Active Ingredients |
|---|---|
| Adrenergic agonist | Adrafinil |
| | Isometheptene |
| | Ephedrine (all forms) |
| Adrenergic antagonist | Monatepil maleate |
| | Naftopidil |
| | Carvedilol |
| | Moxisylyte HCl |
| Adrenergic - | Oxymetazoline HCl |
| Vasoconstrictor/Nasal | Norfenefrine HCl |
| decongestant | Bretylium Tosylate |
| Adrenocorticotropic hormone | Corticotropin |
| Analgesic | Bezitramide |
| | Acetylsalicysalicylic acid |
| | Propanidid |
| | Lidocaine |
| | Pseudophedrine hydrochloride |
| | Acetominophen |
| | Chlorpheniramine Maleate |
| Anesthetics | Dyclonine HCl |
| | Hydroxydione Sodium |
| | Acetamidoeugenol |
| Anthelmintics | Niclosamide |
| | Thymyl N-Isoamylcarbamate |
| | Oxamniquine |
| | Nitroxynil N-ethylglucamine |
| | Anthiolimine |
| | 8-Hydroxyquinoline Sulfate |
| Anti-inflammatory | Bendazac |
| | Bufexamac |
| | Desoximetasone |
| | Amiprilose HCl |
| | Balsalazide Disodium Salt |
| | Benzydamine HCl |
| Antiallergic | Fluticasone propionate |
| | Pemirolast Postassium salt |
| | Cromolyn Disodium salt |
| | Nedocromil Disodium salt |
| Antiamebic | Cephaeline |
| | Phanquinone |
| | Thiocarbarsone |
| Antianemic | Folarin |
| | Calcium folinate |
| Antianginal | Verapamil |
| | Molsidomine |
| | Isosorbide Dinitrate |
| | Acebutolol HCl |
| | Bufetolol HCl |
| | Timolol Hydrogen maleate salt |
| Antiarryhythmics | Quinidine |
| | Lidocaine |
| | Capobenic Acid |
| | Encainide HCl |
| | Bretylium Tosylate |
| | Butobendine Dichloride |
| Antiarthritics | Azathioprine |
| | Calcium 3-aurothio-2-propanol-1-sulfate |
| | Glucosamine Beta Form |
| | Actarit |
| Antiasthmatics/Leukotriene | Cromalyn Disodium |
| antagonist | Halamid |
| | Montelukast Monosodium salt |
| Antibacterial | Cefoxitin Sodium salt |
| | Lincolcina |
| | Colisitin sulfate |
| Antibiotics | Gentamicin |
| | Erythromycin |
| | Azithromycin |
| Anticoagulants | Heprin sodum salt |
| | Heprinar |
| | Dextran Sulfate Sodium |
| Anticonvulsants | Paramethadione |
| | Phenobarbital sodium salt |
| | Levetiracetam |
| Antidepressants | Fluoxetine HCl |
| | Paroxetine |
| | Nortiptyline HCl |
| Antidiabetic | Acarbose |
| | Novorapid |
| | Diabex |
| Antiemetics | Chlorpromazine HCl |
| | Cyclizine HCl |
| | Dimenhydrinate |
| Antiglaucoma agents | Dorzolamide HCl |
| | Epinepherine (all forms) |
| | Dipivefrin HCl |
| Antihistamines | Histapyrrodine HCl |
| Antihyperlipoproteinemic | Lovastatin |
| | Pantethine |
| Antihypertensives | Atenolol |
| | Guanabenz Monoacetate |
| | Hydroflumethiazide |
| Antihyperthyroid | Propylthiouracil |
| | Iodine |
| Antihypotensive | Cortensor |
| | Pholedrine Sulfate |
| | Norepinephrine HCl |
| Antimalarials | Cinchonidine |
| | Cinchonine |
| | Pyrimethamine |
| | Amodiaquin Dihydrochloride dihydrate |
| | Bebeerine HCl |
| | Chloroquine Diphosphate |
| Antimigraine agents | Dihydroergotamine |
| | Ergotamine |
| | Eletriptan Hydrobromide |
| | Valproic Acid Sodium salt |
| | Dihydroergotamine mesylate |
| Antineoplastic | 9-Aminocamptothecin |
| | Carboquone |
| | Benzodepa |
| | Bleomycins |
| | Capecitabine |
| | Doxorubicin HCl |
| Antiparkinsons agents | Methixene |
| | Terguride |
| | Amantadine HCl |
| | Ethylbenzhydramine HCl |
| | Scopolamine N-Oxide Hydrobromide |
| Antiperistaltic; | Bismuth Subcarbonate |
| antidiarrheal | Bismuth Subsalicylate |
| | Mebiquine |
| | Diphenoxylate HCl |
| Antiprotozoal | Fumagillin |
| | Melarsoprol |
| | Nitazoxanide |
| | Aeropent |
| | Pentamideine Isethionate |
| | Oxophenarsine Hydrochloride |
| Antipsycotics | Chlorprothixene |
| | Cyamemazine |
| | Thioridazine |
| | Haloperidol HCl |
| | Triflupromazine HCl |
| | Trifluperidol HCl |
| Antipyretics | Dipyrocetyl |
| | Naproxen |
| | Tetrandrine |
| | Imidazole Salicylate |
| | Lysine Acetylsalicylate |
| | Magnesium Acetylsalicylate |
| Antirheumatic | Auranofin |
| | Azathioprine |
| | Myoral |
| | Penicillamine HCl |
| | Chloroquine Diphosphate |
| | Hydroxychloroquine Sulfate |
| Antispasmodic | Ethaverine |
| | Octaverine |
| | Rociverine |
| | Ethaverine HCl |
| | Fenpiverinium Bromide |
| | Leiopyrrole HCl |
| Antithrombotic | Plafibride |
| | Triflusal |
| | Sulfinpyrazone |
| | Ticlopidine HCl |
| Antitussives | Anethole |
| | Hydrocodone |
| | Oxeladin |
| | Amicibone HCl |
| | Butethamate Citrate |
| | Carbetapentane Citrate |
| Antiulcer agents | Polaprezinc |
| | Lafutidine |
| | Plaunotol |
| | Ranitidine HCl |
| | Pirenzepine 2 HCl |
| | Misoprostol |
| Antiviral agents | Nelfinavir |
| | Atazanavir |
| | Amantadine |
| | Acyclovir |
| | Rimantadine HCl |
| | Epivar |
| | Crixivan |
| Anxiolytics | Alprazolam |
| | Cloxazolam |
| | Oxazolam |
| | Flesinoxan HCl |
| | Chlordiazepoxide HCl |
| | Clorazepic Acid Dipotassium salt |
| Broncodialtor | Epinephrine |
| | Theobromine |
| | Dypylline |
| | Eprozinol 2HCl |
| | Etafedrine |
| Cardiotonics | Cymarin |
| | Oleandrin |
| | Docarpamine |
| | Digitalin |
| | Dopamine HCl |
| | Heptaminol HCl |
| Cholinergic | Eseridine |
| | Physostigmine |
| | Methacholine Chloride |
| | Edrophonium chloride |
| | Juvastigmin |
| Cholinergic antagonist | Pehencarbamide HCl |
| | Glycopyrrolate |
| | Hyoscyamine Sulfate dihydrate |
| Cognition | Idebenone |
| enhancers/Nootropic | Tacrine HCl |
| | Aceglutamide Aluminum Complex |
| | Acetylcarnitine L HCl |
| Decongestants | Propylhexedrine dl-Form |
| | Pseudoephedrine |
| | Tuaminoheptane |
| | Cyclopentamine HCL |
| | Fenoxazoline HCl |
| | Naphazoline HCl |
| Diagnostic aid | Disofenin |
| | Ethiodized Oil |
| | Fluorescein |
| | Diatrizoate sodium |
| | Meglumine Diatrizoate |
| Diuretics | Bendroflumethiazide |
| | Fenquizone |
| | Mercurous Chloride |
| | Amiloride HCl 2 H₂O |
| | Manicol |
| | Urea |
| Enzyme inhibitor | Gabexate Methanesulfonate |
| (proteinase) | |
| Fungicides | Candicidin |
| | Filipin |
| | Lucensomycin |
| | Amphotericin B |
| | Caspofungin Acetate |
| | Viridin |
| Gonad stimulating principle | Clomiphene Citrate |
| | Chorionic gonadotropin |
| | Humegon |
| | Luteinizing hormone (LH) |
| Hemorheologic agent | Poloxamer 331 |
| | Azupentat |
| Hemostatic | Hydrastine |
| | Alginic Acid |
| | Batroxobin |
| | 6-Aminohexanoic acid |
| | Factor IX |
| | Carbazochrome Salicylate |
| Hypolimpemic agents | Clofibric Acid Magnesium salt |
| | Dextran Sulfate Sodium |
| | Meglutol |
| Immunosuppresants | Azathioprine |
| | 6-Mercaptopurine |
| | Prograf |
| | Brequinar Sodium salt |
| | Gusperimus Trihydrochloride |
| | Mizoribine |
| | Rapamycin and analogs thereof |
| Mydriatic; antispasmodic | Epinephrine |
| | Yohimbine |
| | Aminopentamide dl-Form |
| | Atropine Methylnitrate |
| | Atropine Sulfatemonohydrate |
| | Hydroxyamphetamine (I, HCl, HBr) |
| Neuromuscular blocking | Phenprobamate |
| agent/Muscle relaxants | Chlorzoxazone |
| (skeletal) | Mephenoxalone |
| | Mioblock |
| | Doxacurium Chloride |
| | Pancuronium bromide |
| Oxotocic | Ergonovine Tartrate hydrate |
| | Methylergonovine |
| | Prostaglandin F_{2α} |
| | Intertocine-S |
| | Ergonovine Maleate |
| | Prostoglandin F_{2α} Tromethamine salt |
| Radioprotective agent | Amifostine 3H₂O |
| Sedative/Hypnotic | Haloxazolam |
| | Butalbital |
| | Butethal |
| | Pentaerythritol Chloral |
| | Diethylbromoacetamide |
| | Barbital Sodium salt |
| Serenic | Eltoprazine |
| Tocolytic agents | Albuterol Sulfate |
| | Terbutaline sulfate |
| Treatment of cystic fibrosis | Uridine 5′-Triphosphate Trisodium |
| | dihydrate salt |
| Vasoconstrictor | Nordefrin (−) Form |
| | Propylhexedrine dl-form |
| | Nordefrin HCl |
| Vasodilators | Nylidrin HCl |
| | Papaverine |
| | Erythrityl Tetranitrate |
| | Pentoxifylline |
| | Diazenium diolates |
| | Citicoline |
| | Hexestrol Bis(β-diethylaminoethyl ether) |
| | 2HCl |
| Vitamins | α-Carotene |
| | β-Carotene |
| | Vitamin D₃ |
| | Pantothenic Acid sodium salt |

Other desirable therapeutic agents include, but are not limited to, the following: (a) anti-inflammatory/immunomodulators such as dexamethasone, m-prednisolone, interferon g-1b, leflunomide, sirolimus, tacrolimus, everolimus, pimecrolimus, biolimus (such as Biolimus A7 or A9) mycophenolic acid, mizoribine, cyclosporine, tranilast, and viral proteins; (b) antiproliferatives such as paclitaxel or other taxane derivatives (such as QP-2), actinomycin, methothrexate, angiopeptin, vincristine, mitomycine, statins, C MYC antisense, ABT-578, RestenASE, Resten-NG, 2-chloro-deoxyadenosine, and PCNA ribozyme; (c) migration inhibitors/ECM-modulators such as batimastat, prolyl hydroxylase inhibitors, halofuginone, C proteinase inhibitors, and probucol; and (d) agents that promote healing and re-endothelialization such as BCP671, VEGF, estradiols (such as 17-beta estradiol (estrogen)), NO donors, EPC antibodies, biorest, ECs, CD-34 antibodies, and advanced coatings. Any single bioactive agent or combination of bioactive agents may be used in the present invention. Preferred bioactive agents used with the implantable medical devices of the invention can be, for example, drugs useful for pain management, antiproliferative agents (e.g. paclitaxel, or pharmaceutically acceptable salts, esters or prodrugs thereof), anticancer drugs, insulin (or pharmaceutically acceptable salts, esters or prodrugs thereof), medications to regulate levels of neurotransmitters (e.g., serotonin and dopamine) in the brain, thereby treating psychological conditions such as, for example, depression or attention deficit hyperactivity disorder and nitric oxide-containing compounds for the treatment of a range of disorders, including, for example, erectile disfunction, septic shock, and stroke.

The desired loading level or concentration of the bioactive agent in the nanocomposite coating may vary over a broad range. Preferably, the concentration of bioactive agent in the nanocomposite coating will range from about 0.1% to about 50% by volume. More preferably, the concentration of bioactive agent may range from about 0.5% to about 40% by volume. Even more preferably, the concentration of bioactive agent may range from about 1% to about 30% by volume.

The nanocomposite coating described herein is deposited on at least a portion of a surface of at least one structural element of an implantable medical device. The device has a structure comprising one or more of the structural elements. The structure is adapted to interact mechanically or electrically with tissue or other body part or constituent. That mechanical interaction may involve the application of a force to open or maintain open a lumen or to hold body parts together or in a defined mutual relationship. That mechanical interaction may be filtering or the physical promotion of clotting; occlusion of a vessel or vessel portion; or the creation, maintenance or repair of a fluid-tight seal in the body. That structure may be adapted to remain essentially permanently within the body or at least to remain within the body for a time period which is very long in comparison with the time period over which the bioactive agent is adapted to be released. That structure may be wholly or in part non-biodegradable or at least biodegradable at a rate which is very slow in comparison with the rate at which bioactive material is adapted to be released. The structure may be formed wholly or in part from metal.

Preferably, the medical device is a prosthesis. The medical device may be, for example, a stent, stent graft, vascular graft, catheter, guide wire, balloon, filter (e.g., vena cava filter), cerebral aneurysm filler coil, intraluminal paving system, suture, staple, anastomosis device, vertebral disk, bone pin, suture anchor, hemostatic barrier, clamp, screw, plate, clip, sling, vascular implant, tissue adhesive or sealant, tissue scaffold, myocardial plug, pacemaker lead, valve (e.g. venous valve), abdominal aortic aneurysm (AAA) graft, embolic coil, dressing, bone substitute, intraluminal device, vascular support or other known biocompatible device. Examples of stents that may be used in the present invention include self-expandable or balloon-expandable stents, including endovascular, biliary, tracheal, gastrointestinal, urethral, ureteral, esophageal and coronary vascular stents.

The structural element having the surface upon which the nanocomposite coating is applied may be any element of the device which, when implanted, is exposed to or otherwise communicates with tissue or another body part or constituent that will benefit from the bioactive agent. For example, when the device is a coronary stent, the structural element is preferably a strut. In another example, when the device is a filter, the structural element is preferably a wire. In yet another example, when the device is a catheter, the structural element is preferably a tubular body.

According to some embodiments, the one or more structural elements of the implantable medical device may be made of at least one of: stainless steel, nitinol, gold, silver, tantalum, platinum, iridium, niobium, tungsten, titanium, cobalt, chromium, magnesium, aluminum, nickel, or another biocompatible metal or alloy; cellulose acetate, cellulose nitrate, silicone, cross-linked polyvinyl alcohol (PVA) hydrogel, polyurethane, polyamide, styrene isobutylene-styrene block copolymer, polyethylene teraphthalate, polyester, polyorthoester, polyanhydride, polyethersulfone, polycarbonate, polypropylene, high molecular weight polyethylene, polytetrafluoroethylene, or another biocompatible polymeric material, or mixtures or copolymers of these; polylactic acid, polyglycolic acid or copolymers thereof, a polyanhydride, polycaprolactone, polyhydroxybutyrate valerate or another biodegradeable polymer, or mixtures or copolymers of these; carbon or carbon fiber; ceramic materials, such as, for example, calcium phosphate; a protein, extracellular matrix component, collagen, fibrin, or another biologic agent; or a suitable mixture of any of these.

Any surface or portion of a surface of a structural element of an implantable medical device may be coated with the nanocomposite coating. The surface may be flat or curved, smooth or rough, or some combination thereof. A “rough” surface may be, for example, textured, woven, or non-woven, and/or contain channels, recesses, indentations, projections, ridges, or similar features.

It may be advantageous to prepare the surface of the structural element before depositing the nanocomposite coating thereon. Useful methods of surface preparation may include, but are not limited to: cleaning, etching, drilling, abrasion, plasma treatment, and ion bombardment. Such surface preparation may activate the surface and promote the deposition or adhesion of the coating on the surface.

Preferably, from about 20% to about 100% of the surface may be coated with the nanocomposite coating. More preferably, from about 40% to about 100% of the surface may be coated. Even more preferably, from about 60% to about 100% of the surface may be coated with the nanocomposite coating.

A method for preparing an implantable medical device coated with a nanocomposite coating for the controlled release of a bioactive agent is also described. According to one embodiment of the method, a nanocomposite coating formulation including inorganic particles and a bioactive agent may be prepared by combining liquid precursors of the matrix with the inorganic particles and bioactive agent, and then mixing. In a next step, the coating formulation may be deposited onto at least a portion of a surface of an implantable medical device, thereby forming a coated medical device with a nanocomposite coating. A variety of coating methods are known in the art and may be used to deposit the coating formulation, such as, for example, dip coating, bar coating, spray coating, or spin coating. Preferably, the coating formulation is applied to the outer surface of the medical device. If necessary, the coating may be cured. Curing may be carried out by any method known in the art, for example, by application of heat or exposure to radiation, such as ultraviolet (UV) radiation. A biocompatible coating may further be deposited on the nanocomposite coating.

According to another embodiment of the method, a first coating formulation may be prepared by combining liquid precursors of the matrix with the inorganic particles and then mixing. A second coating formulation may be prepared by combining a bioactive agent with liquid precursors of the matrix and then mixing. In a next step, the first and second coating formulations may be sequentially deposited onto at least a portion of a surface of an implantable medical device, thereby forming a coated medical device with a nanocomposite coating. A variety of coating methods are known in the art and may be used to deposit the coating formulations, such as, for example, dip coating, bar coating, spray coating, or spin coating. Preferably, the coating formulations are applied to the outer surface of the medical device. Curing of the coating formulations may be carried out by any method known in the art, for example, by application of heat, chemicals, or exposure to radiation, such as ultraviolet (UV) radiation. A biocompatible coating may further be deposited on the nanocomposite coating.

Alternatively, the second coating formulation may not be formed and a bioactive agent may be loaded into the nanocomposite coating directly after application of the first coating formulation to the medical device. Loading of the bioactive agent into the nanocomposite coating may be achieved by, for example, diffusion into the matrix or (re)hydration in the desired bioactive solutions.

A method of providing a controlled release of a bioactive agent from a nanocomposite coating on an implantable medical device is also provided. The method includes inserting a medical device having a nanocomposite coating disposed on at least a portion of a surface of at least one structural element of the device into a body lumen. The coated medical device may be deployed in a vessel within the body using standard deployment techniques known to medical professionals. For example, according to an embodiment in which the coated medical device is a self-expandable stent comprising a nanocomposite coating on an outer surface of one or more struts of the stent, the stent may be mounted within a retaining sheath which contacts the outer surface of the stent and retains the stent in a compressed state for delivery into a vessel. A hollow needle may be used to penetrate the vessel, and a guide wire may be threaded through the needle into the vessel. The needle may then be removed and replaced with an introduction catheter, which generally acts as a port through which stents and other medical devices may then be passed to gain access to a vessel. Once the stent and retaining sheath are passed through the introduction catheter and positioned within the vessel adjacent to the site to be treated, the retaining sheath may be retracted, thereby causing the stent to expand from the compressed state to an expanded state. In the expanded state, the outer surface of the stent contacts and exerts a radial force on the vessel wall. The retaining sheath and the introduction catheter may then be withdrawn from the vessel. Other standard deployment techniques may be used to insert other types of coated medical devices.

Once the medical device is implanted, the inorganic particles may be exposed to a stimulus, preferably electromagnetic radiation or a magnetic field, that causes at least a portion of the bioactive agent to be released from the surface of the coated medical device. The bioactive agent is substantially not released until exposure to the stimulus occurs. A first exposure to the stimulus may be provided immediately upon implantation of the device, or at a later time. Preferably, the later time may range from about several minutes to several years after the medical device is implanted. More preferably, the later time may range from about one hour to about six months after implantation. Even more preferably, the later time may range from about one day to about one month after implantation. When the stimulus is removed, release of the bioactive agent is substantially halted.

One or more additional exposures to the stimulus may occur following the first exposure to the stimulus in order to release multiple dosages of the bioactive agent. These additional exposures may occur at any time after the first exposure and before the expiration of five years from implantation of the device.

Each exposure to the stimulus may be instantaneous, or the exposure to the stimulus may occur over a measurable duration of time. This duration of time may range from about one second to about 90 minutes. Preferably, the duration of each exposure ranges from about one minute to about 60 minutes. Even more preferably, the duration of each exposure ranges from about five minutes to about 45 minutes.

The present method may allow for the controlled release of a bioactive agent in one or more or more dosages from an implantable medical device having a nanocomposite coating.

For example, according to one embodiment, a ureteral stent having a nanocomposite coating may be used to controllably release a drug for pain management following ureteroscopy.

According to another embodiment, a stent or stent graft having a nanocomposite coating may be used to treat restenosis in a blood vessel by controllably releasing an antiproliferative agent such as, for example, paclitaxel.

According to another embodiment, a stent or stent graft having a nanocomposite coating may be used to treat a malignant tumor in the bile duct by controllably releasing an anticancer drug, such as, for example, paclitaxel.

According to another embodiment, an implantable medical device having a nanocomposite coating may be used to controllably release insulin for the treatment of diabetes.

According to another embodiment, an implantable medical device having a nanocomposite coating may be used to controllably release medications to regulate levels of neurotransmitters (e.g., serotonin and dopamine) in the brain, thereby treating psychological conditions such as, for example, depression or attention deficit hyperactivity disorder (ADHD).

According to another embodiment, a medical device having a nanocomposite coating may be used to controllably release nitric oxide-containing compounds for the treatment of a range of disorders, including, for example, erectile disfunction, septic shock, and stroke.

Although the present invention has been described in considerable detail with reference to certain embodiments thereof, other embodiments are possible without departing from the present invention. The spirit and scope of the appended claims should not be limited, therefore, to the description of the preferred embodiments contained herein. All devices and methods that come within the meaning of the claims, either literally or by equivalence, are intended to be embraced therein. It is intended that the foregoing detailed description be regarded as illustrative rather than limiting, and that it be understood that it is the following claims, including all equivalents, that are intended to define the scope of this invention.

What is claimed is:

1. A medical device for performing a function when implanted within an animal and for providing a controlled release of a bioactive agent, the medical device comprising:
at least one structural element including a surface;
a nanocomposite coating deposited on at least a portion of the surface, wherein the nanocomposite coating comprises a matrix, a bioactive agent, and inorganic particles, the inorganic particles being responsive to a stimulus; and
wherein at least a portion of the bioactive agent is released from the nanocomposite coating when the inorganic particles are exposed to the stimulus.

2. The medical device according to claim 1, wherein the matrix comprises a polymer.

3. The medical device according to claim 2, wherein the polymer is biodegradable.

4. The medical device according to claim 2, wherein the polymer is a hydrogel.

5. The medical device according to claim 4, wherein the hydrogel is poly(N-isopropylacrylamide).

6. The medical device according to claim 1, wherein the nanocomposite coating comprises two or more layers.

7. The medical device according to 1, wherein a biocompatible layer is disposed on the nanocomposite coating.

8. The medical device according to claim 1, wherein the coating has a thickness of from about 0.1 micron to about 100 microns.

9. The medical device according to claim 1, wherein the inorganic particles are exposed to the stimulus more than once over a period of time, thereby releasing the bioactive agent in multiple dosages.

10. The medical device according to claim 1, wherein the inorganic particles comprise at least one element selected from the group consisting of Au, Ag, Pt, Pd, Ir, Rh, Ru, Os, Re, Tc, W, Ta, Nb, Hf, Zr, Y, Sc, Ti, V, Cr, Mo, Mn, Tc, Fe, Co, Ni, Cu, Zn, Cd, Al, Ga, In, Tl, Si, Ge, Sn, Pb, Bi, Sb, As, Se, Te, Po, Ce, Pr, Nd, Sm, Eu, Gd, Th, Dy, Ho, Er, Tm, Yb, and Lu.

11. The medical device according to claim 1, wherein the inorganic particles are about 100 nanometers or less in size.

12. The medical device according to claim 1, wherein the inorganic particles have a core-shell structure comprising a core and an outer layer surrounding the core.

13. The medical device according to claim 1, wherein the inorganic particles generate heat in response to the stimulus.

14. The medical device according to claim 1, wherein the stimulus is electromagnetic radiation.

15. A medical device for performing a function when implanted within an animal and for providing a controlled release of a bioactive agent, the medical device comprising:
at least one structural element including a surface;
a nanocomposite coating deposited on the surface, wherein the nanocomposite coating comprises a hydrogel, a bioactive agent, and metal nanoshells, the metal nanoshells being responsive to electromagnetic radiation; and
wherein at least a portion of the bioactive agent is released from the nanocomposite coating when the metal nanoshells are exposed to the electromagnetic radiation.

16. A method for providing a controlled release of a bioactive agent from a medical device having a function when implanted within an animal, the method comprising:
inserting an implantable medical device comprising a nanocomposite coating on at least a portion of a surface of at least one structural element of the device into a body lumen, wherein the nanocomposite coating comprises a matrix, a bioactive agent, and inorganic particles, the inorganic particles being responsive to a stimulus;
exposing the inorganic particles to the stimulus during a first exposure, thereby causing at least a portion of the bioactive agent to be released from the nanocomposite coating.

17. The method of claim 16, further comprising exposing the inorganic particles to the stimulus during one or more additional exposures.

18. The method of claim 17, wherein each of the first exposure and the one or more additional exposures has a duration of from about 1 minute to about 90 minutes.

19. A method for providing a coating for the controlled release of a bioactive agent on a medical device having a function when implanted within an animal, the method comprising:
preparing a coating formulation comprising a matrix precursor and inorganic particles, the inorganic particles being responsive to a stimulus;
depositing the coating formulation onto at least a portion of a surface of at least one structural element of an implantable medical device, thereby forming a coated implantable medical device having a nanocomposite coating.

20. The method according to claim 19, wherein the coating formulation further comprises a bioactive agent.

21. The method according to claim 19, further comprising:
loading a bioactive agent into the nanocomposite coating.

22. A method for providing a coating for the controlled release of a bioactive agent on a medical device having a function when implanted within an animal, the method comprising:
preparing a first coating formulation comprising a first matrix precursor and inorganic particles, the inorganic particles being responsive to a stimulus;
preparing a second coating formulation comprising a second matrix precursor and a bioactive agent;
sequentially depositing the first coating formulation and the second coating formulation onto at least a portion of a surface of at least one structural element of an implantable medical device, thereby forming a coated implantable medical device having a nanocomposite coating.
